(19) United States
(12) Patent Application Publication
(10) Pub. No.: US 2007/0021804 A1
(43) Pub. Date: Jan. 25, 2007

(54) Stimulation using a microstimulator to treat tinnitus

(76) Inventors: Albert A. Maltan, Sistrans (AT); Todd K. Whitehurst, Santa Clarita, CA (US)

Correspondence Address: ADVANCED BIONICS CORPORATION, 25129 RYE CANYON ROAD, VALENCIA, CA, 91355, US

(21) Appl. No.: 10/854,108

(22) Filed: May 26, 2004

Related U.S. Application Data
(60) Provisional application No. 60/474,505, filed on May 30, 2003.

(51) Int. Cl. A61N 1/00

(52) U.S. Cl. 607/55

(57) ABSTRACT

An implantable microstimulator and a stimulating electrode are implanted to treat tinnitus. The microstimulator is connected to the stimulating electrode via a short, flexible lead. The electrode is implanted in the promontory of the cochlea or in the cochlear round window niche to deliver electrical stimulation to the auditory nervous system and thereby suppress or mask the tinnitus. The microstimulator may be surgically implanted through the ear canal, via a mastoidectomy or through the middle fossa, i.e., a supra-meatal approach. The microstimulator may be placed in middle ear cavity, a bony recess created in the bone surrounding the middle ear, the mastoid, the cochlea or a part of the temporal bone. In addition, the system may further include a test electrode, a test stimulator and a remote programming device that employs a bi-directional radio-frequency communication link to control and program the microstimulator and the test stimulator.

The present application claims the benefit of United States Provisional Patent Application Ser. No. 60/474,505, filed 30 May 2003, which application is herein incorporated by reference in its entirety.

### BACKGROUND OF THE INVENTION

The present invention relates to treatment of tinnitus and, more particularly, relates to treatment of tinnitus using a microstimulator that emits electrical stimulation in the auditory system to suppress or mask the perception of extraneous sounds associated with tinnitus.

Although “tinnitus aureum” literally means in Latin “ringing of the ears,” the term “tinnitus” is commonly used to encompass a variety of extraneous noises (which does not arise from an outside source) including buzzing, humming, whistling and roaring sounds. Tinnitus is a common hearing disorder affecting an estimated 15 per cent of the U.S. population according to the National Institutes of Health, 1989 Research Plan. Extrapolating this percentage, approximately 9 million Americans suffer from at least a mild case of tinnitus and, of those, about 2 million have a severe form of tinnitus that may be characterized, in many cases, as debilitating.

Various treatments have been used to treat or curtail the occurrence of tinnitus. One treatment employs acoustic maskers. Acoustic maskers “mask” the perception of undesirable sounds by presenting alternative sounds produced by an external sound generator. U.S. Pat. Nos. 5,325,872, 5,403,262 and 5,788,656 provide some examples of treatments using acoustic maskers. Other forms of treatments include bio-feedback, acupuncture and application of medications. The many forms of treatments for tinnitus attest to the uneven results achieved by these treatments. Most of these treatments, unfortunately, do not provide relief for the large percentage of those who suffer from severe tinnitus.

Accordingly, what is needed is a device and method that provide an alternative treatment for patients who do not respond to current treatments for tinnitus.

### SUMMARY OF THE INVENTION

The present invention addresses the above and other needs by providing a system and method that treats tinnitus.

In an aspect of the present invention, a stimulation system is provided which includes a fully implantable microstimulator and electrode, which system is essentially non-obtrusive once implanted.

An embodiment of the stimulation system includes not only the implantable microstimulator and electrode, but also a temporary electrode and a test stimulator. The latter two devices are used to pre-screen the patient to ensure that the stimulation therapy being provided is effective, i.e., that the tinnitus is being masked or suppressed. In addition, if the tinnitus is being adequately treated, the current location of the temporary electrode is confirmed to be satisfactory and the permanent electrode may be implanted in the same location. It may be necessary to place the temporary electrode in several sites to identify the best electrode placement. The preliminary testing with the temporary electrode may take place over a period of hours, days or even weeks before making the commitment to implant the permanent electrode. The system may further comprise a remote control device that can control the permanent microstimulator, the test stimulator or both stimulators.

In another aspect of the invention, a method is provided for treating tinnitus in which the perception of extraneous sounds is suppressed or masked by electrically stimulating the auditory system, particularly at the promontory of the cochlea or at the round (cochlear) window.

In one embodiment, a method is provided in which a microstimulator and an electrode are implanted, whereby the system is completely implantable in the auditory system, owing to the small size of the entire stimulation system. A further embodiment of the method includes providing a test stimulation by using a temporary electrode and applying electrical stimulation through the electrode using a test stimulator. A remote control device having a bi-directional, communications link may be used to program and receive data from the test stimulator, the microstimulator or both.

It is thus a feature of the present invention that a complete microstimulator system may be advantageously implanted in the auditory system which, otherwise, has little room to accommodate conventionally sized, implantable devices. The microstimulator may have a rechargeable battery that allows recharging of the battery through the skin using an external device that transfers radio-frequency (RF) energy.

Another feature of the present invention is that a pre-testing procedure can be used to improve the efficacy of the therapy by confirming that the electrode placement is good and that stimulation is effectively treating tinnitus before implanting the permanent system.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows an illustration of the human auditory system;

FIG. 2 shows, in accordance with the present invention, an illustration of a microstimulator system for treating tinnitus, which mircrostimulator has an electrode attached via a short lead;

FIG. 3 shows, in accordance with the present invention, a depiction of the electrode, placed in exemplary locations in the auditory system, in particular, the promontory of the cochlea and the round window niche, while the microstimulator (not shown in FIG. 3), may be placed in the middle ear cavity, the bony recess created in the bone surrounding the middle ear, the mastoid, the cochlea or another part of the temporal bone; and

FIG. 4 shows the test system comprising a temporary electrode, a test (non-implanted) stimulator and an optional, remote programming device that may bi-directionally communicate with the test stimulator or with the permanently implanted microstimulator shown in FIG. 2.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

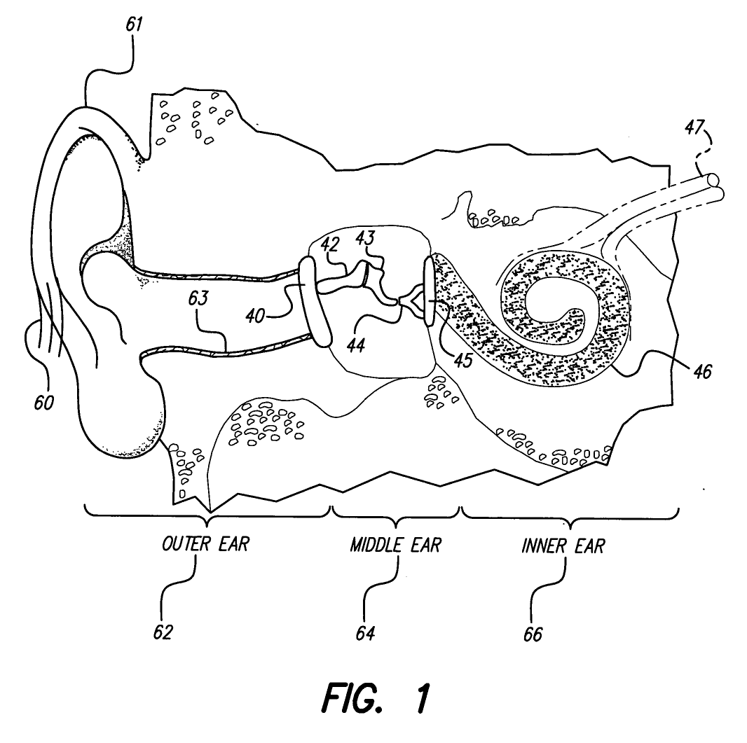

FIG. 1 shows a representation of a human auditory system divided into the outer, middle and inner ear. It is instructive to review the operation of the normal auditory apparatus in order to discuss the treatment for tinnitus. As shown, sound waves 60 are collected by the auricle 61 of the outer ear 62. The collected sound waves 60 then pass through the ear canal 63 and strike the eardrum or typmpanic membrane 40 causing it to vibrate in accordance with the frequencies and intensity of the sound waves 60. In a functioning ear, this creates a chain reaction in the three tiny bones in the middle ear 64, which bones are the malleus 42, the incus 43 and stapes 44. Movements of these bones, in turn, generate movement of the round window 45, causing movement in the fluid contained in the cochlea 46, located in the inner ear 66. The fluid movement causes hair cells contained within the snail-shaped cochlea to deflect. Deflection of the hair cells causes ganglion nerves located at the base of the hair cells to transmit electrical signals coded for the frequencies and intensity of the sound waves 60. The individual ganglion nerves converge together as the auditory nerve 47 which enters the brain. Nerve impulses course through the auditory nerve, which impulses are coded for sound frequencies and intensity and decoded in the brain, resulting in the perception of different sounds.

While the cause or causes of tinnitus is not well understood, it is believed that certain types of tinnitus, for example, a ringing consisting of specific sound frequencies, occur because groups of nerve cells in the cochlea coding for the specific sound frequencies are being stimulated by something other than the externally sourced sound waves 60. It is not known what causes this extraneous stimulation of the specific nerve cells in the pattern perceived as ringing or, for that matter, other types of sounds broadly referred to as tinnitus. Because tinnitus may result from dysfunctional stimulation of specific auditory nerves, the present invention uses a system and method to suppress or mask the tinnitus resulting from such dysfunctional nerve stimulation.

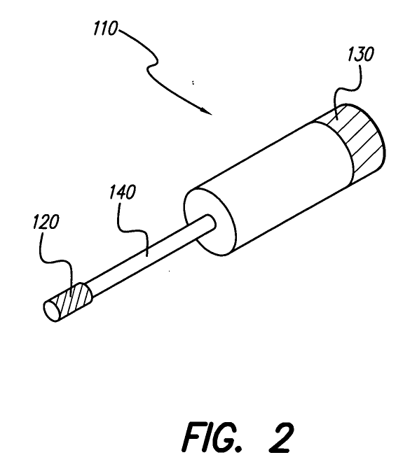

FIG. 2 shows, in accordance with the present invention, a simplified representation of a stimulation system that includes a microstimulator 110 and electrode 120. The microstimulator 110 may have an exterior form that is approximately cylindrical. The microstimulator is electrically connected to a stimulating electrode 120 that is placed at the distal end of a short lead 140. A second, indifferent electrode 130 may be placed somewhere on the housing of the microstimulator 110. In one embodiment, substantially the entire housing of the microstimulator 110 may be formed of a conductive material and act as an indifferent electrode 130. The indifferent electrode 130 may be made of body compatible, electrically conductive material such as titanium or a titanium alloy and the use of such materials may also ensure the hermeticity of the microstimulator. The electrode 120 may be made from known, biocompatible electrode materials such as platinum, platinum/iridium, gold, carbon and other commonly used electrode materials that may be used within a corrosive, body environment. The short lead extension 140, which is preferably flexible, encloses a conductor material with an outer insulation that may be made from implantable grade silicone or polyurethane. The microstimulator 110 in the preferred embodiment is less than three centimeters long and preferably assumes a substantially cylindrical outer form. The diameter of the microstimulator is preferably less than one centimeter. The lead extension 140 preferably ranges from about 1 to 10 centimeters in length, although it may be made longer to accommodate more distant placement of the microstimulator 110 from the electrode 120.

An example of a suitable microstimulator is the BION® microstimulator manufactured by Advanced Bionics Corporation, Sylmar, California. U.S. patent application Ser. No. 09/624,130, filed Jul. 24, 2000 and U.S. patent application Ser. No. 10/607,963, filed Jun. 27, 2003, describe embodiments of the BION® microstimulator in more detail, including construction of the microstimulator and the inner circuitry, as well as the operation of the microstimulator. Both applications are herein incorporated by reference in their entireties.

Other applications/publications/patents, listed in the table below, and which are all herein incorporated by reference, describe other details associated with the manufacture, operation and use of BION® implantable microstimulators.

TABLE 1

| Application/Patent/ | Filing/Publication | |
|---|---|---|
| Publication No. | Date | Title |
| U.S. Pat. No. 5,193,539 | Issued | Implantable Microstimulator |
| | Mar. 16, 1993 | |
| U.S. Pat. No. 5,193,540 | Issued | Structure and Method of Manufacture of an Implantable |
| | Mar. 16, 1993 | Microstimulator |
| U.S. Pat. No. 5,312,439 | Issued | Implantable Device Having an Electrolytic Storage |
| | May 17, 1994 | Electrode |
| U.S. Pat. No. 5,324,316 | Issued | Implantable Microstimulator |
| | Jun. 28, 1994 | |
| U.S. Pat. No. 5,405,367 | Issued | Structure and Method of Manufacture of an Implantable |
| | Apr. 11, 1995 | Microstimulator |
| U.S. Pat. No. 6,185,452 | Issued | Battery-Powered Patient Implantable Device |
| | Feb. 6, 2001 | |
| U.S. Pat. No. 6,164,284 | Issued | System of Implantable Devices For Monitoring and/or |
| | Dec. 26, 2000 | Affecting Body Parameters |
| U.S. Pat. No. 6,208,894 | Issued | System of Implantable Devices For Monitoring and/or |
| | Mar. 27, 2001 | Affecting Body Parameters |
| U.S. Pat. No. 6,051,017 | Issued | Implantable Microstimulator and Systems Employing the |
| | Apr. 18, 2000 | Same |
| | Published | Micromodular Implants to Provide Electrical Stimulation |
| | September, 1997 | of Paralyzed Muscles and Limbs, by Cameron, et al., |
| | | published in IEEE Transactions on Biomedical |
| | | Engineering, Vol. 44, No. 9, pages 781-790. |

The BION® microstimulator may have a rechargeable battery that can be charged from an external radio-frequency (RF) source. The BION® microstimulator may also have a transceiver that can receive the RF source of energy and convey that energy as an induced alternating current within the microstimulator, which current may be rectified and used to recharge the battery.

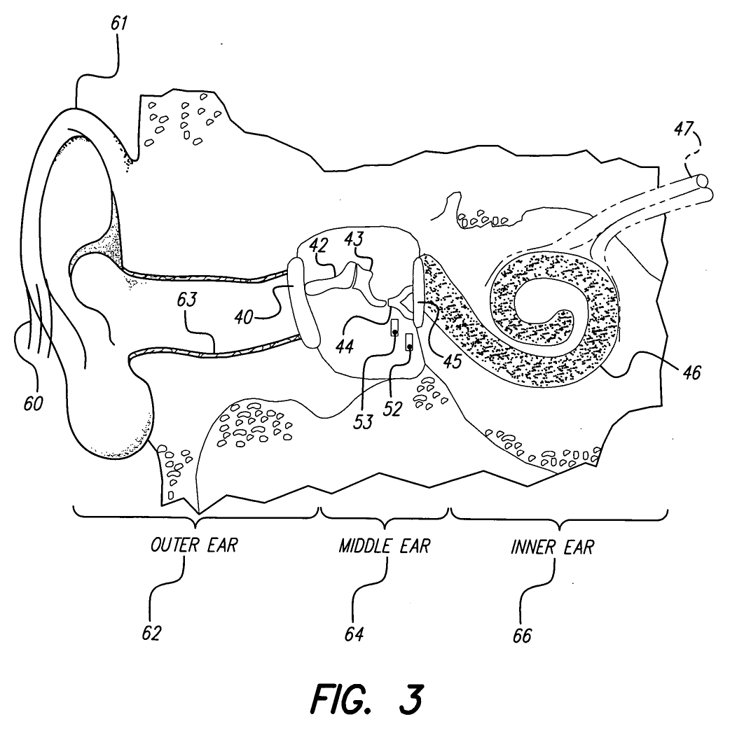

FIG. 3 shows placement of the stimulating electrode 20 in the promontory of the cochlea 53. Alternatively, the stimulating electrode 20 may be implanted in the cochlear round window niche 52. Both locations permit the stimulating electrode to stimulate nerves in the auditory system. The microstimulator may be placed in a suitable cavity in the middle ear 64. Alternative locations for placing the microstimulator include the mastoid, the cochlea 46 and within the temporal bone.

Surgically, the microstimulator and electrode may be placed through a small incision through the ear canal, via a mastoidectomy, or through the middle fossa, i.e., a supra-meatal approach, or through another access that is parallel to the ear canal.

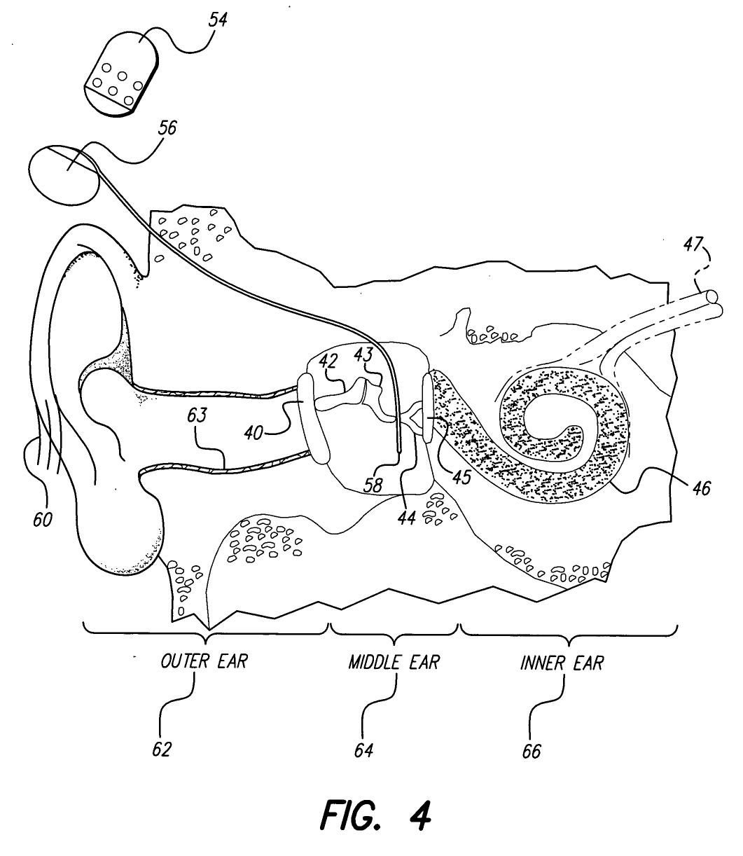

FIG. 4 shows, in accordance with the present invention, a system that is used to preliminarily test a patient by applying electrical stimulation from the test stimulator. The complete test system includes the temporary electrode 58, a non-permanent, test stimulator 56, which is preferably non-implantable and, optionally, an external remote device 54. The remote control device 54 may be used to control and program the non-permanent stimulator 56 using a bidirectional communications link, such as an RF link. Stimulation parameters such as stimulus pulsewidth, stimulus amplitude and frequency of stimulus pulses may be adjusted with the remote control device, by a clinician or a patient. In addition, the remote control device 54 may also be used to control the microstimulator 110 after it is permanently implanted.

In an alternative test system, the test stimulator 56 may have user operated controls and may be programmed using these controls. In that case, a remote device 54 may be unnecessary.

In operation, the temporary electrode 58 can be used to test an electrode placement site and whether a particular electrical stimulation protocol is effective for either masking or suppressing the sounds produced in tinnitus. The temporary electrode 58 is used to find a good electrode placement site, and once found, the permanent electrode 20 should be implanted in the same location. Preferably, the temporary electrode should be placed somewhere in the cochlear promontory or in the cochlear round window niche to stimulate the auditory nervous system and thereby effectively treat tinnitus. The temporary electrode can be placed by opening an incision through the ear canal, through a mastoidectomy, through a middle fossa (supra-meatal) approach, or through an alternative path that is parallel to the ear canal. The test stimulator 56 may be temporarily placed inside the ear canal, worn behind the ear or on some other location on the body.

The temporary electrode and test stimulator may be used over a period of hours, days or weeks and various test stimulation parameters may be tried to assess if the electrical stimulation treatment is adequately suppressing or masking the occurrence of tinnitus. Once therapeutic efficacy is confirmed, the location of the temporary electrode and the stimulation parameters used may be noted and saved. Then the temporary electrode can be removed and the permanent electrode 20 and microstimulator 10 may be implanted. This initial procedural step of implanting a temporary test electrode ensures that the permanent stimulation system is not implanted unless the treatment proves beneficial in advance. This saves the patient from unnecessary subsequent implantation of devices and improves the rate of treatment success.

In summary, the present invention provides a system for treating tinnitus using electrical stimulation comprising: an implantable microstimulator; and an electrode electrically connected to the microstimulator. The electrode may be placed to stimulate the auditory system with electrical stimulation to mask or suppress the perception of tinnitus. In one embodiment of the system, the electrode may be placed on an extending lead that sticks out from the microstimulator and which extending lead has a conductor running through and which conductor connects the electrode with the electronic circuitry in the microstimulator. An external remote device may also be included in the system which remote device has an RF telemetry communications link for bi-directionally communicating with the implantable microstimulator. The microstimulator, in the preferred embodiment, has a rechargeable battery, which is recharged through transcutaneous inductive charging. An external charging unit may be used that transmits radio-frequency (RF) energy that can be picked up by the implantable microstimulator and which energy is used to charge the rechargeable battery. Using the system of the invention, the electrode may be placed in the promontory of the cochlea or, alternatively, the electrode may be placed in the round window niche.

The invention also encompasses a method for treating tinnitus by applying electrical stimulation, and thereby stimulating the auditory system in order to mask or suppress the occurrence of tinnitus. In one embodiment of the invention, the method comprises: (a) implanting a microstimulator, having a stimulation output, into a part of the auditory system; (b) implanting an electrode in a pre-determined location in the auditory system, which electrode is electrically connected to a stimulation output of the microstimulator; and (c) providing stimulation through the electrode to mask or suppress the occurrence of tinnitus.

In one embodiment of the method, the step of implanting a microstimulator may be performed by implanting in one or more of the following ear locations: the middle ear cavity, a bony recess created in the bone surrounding the middle ear, the mastoid, the cochlea and the temporal bone. In another embodiment the step of implanting a microstimulator can comprise one of the following surgical approaches: through the ear canal, through a middle fossa (supra-meatal) approach, and through a mastoidectomy. In yet another embodiment of the method, the following may be performed before step (a): performing a test stimulation procedure comprising: implanting a temporary electrode in the auditory system; connecting temporarily the temporary electrode to a non-implanted stimulator; and providing stimulation through the temporary electrode to determine the efficacy of the temporary electrode location. The non-implanted stimulator may be worn in the inner ear or alternatively, worn behind-the-ear (BTE). As a further embodiment, the non-implanted stimulator may be worn on the body.

As further embodiments to the method described, the step of implanting an electrode system in a pre-determined location in the auditory system may be performed by implanting the electrode in the promontory of the cochlea and/or by implanting the electrode in the round window niche.

While the invention herein disclosed has been described by means of specific embodiments and applications thereof, numerous modifications and variations could be made thereto by those skilled in the art without departing from the scope of the invention set forth in the claims.

1. A system for treating tinnitus using electrical stimulation comprising:
an implantable microstimulator configured to be implanted within a recess created within a bone surrounding the middle ear; and
an electrode electrically connected to the microstimulator;
wherein the electrode is placed to stimulate the auditory system with electrical stimulation to mask or suppress the perception of tinnitus.

2. The system of claim 1, further comprising:
an extending lead coupled to and extending from the microstimulator;
wherein the electrode is placed on the extending lead.

3. The system of claim 1, further comprising:
an external device with RF telemetry for bi-directionally communicating with the implantable microstimulator.

4. The system of claim 1, wherein the microstimulator has a rechargeable battery.

5. The system of claim 4, further comprising:
an external charging unit that transmits radio-frequency (RF) energy that may be picked up by the implantable microstimulator, which energy is used to charge up the rechargeable battery.

6. The system of claim 1, wherein the electrode is placed in the promontory of the cochlea.

7. The system of claim 1, wherein the electrode is placed in the round window niche.

8. A method of treating tinnitus by applying electrical stimulation comprising:
providing a microstimulator and an electrode electrically coupled thereto;
implanting the microstimulator into a recess created within a bone surrounding the middle ear;
stimulating the auditory system with electrical stimulation applied via the electrode to mask or suppress the occurrence of tinnitus.

9. A method for treating tinnitus comprising:
(a) implanting a microstimulator, having a stimulation output, into a recess created within a bone surrounding the middle ear;
(b) implanting an electrode in a pre-determined location in the auditory system, which electrode is electrically connected to a stimulation output of the microstimulator; and
(c) providing stimulation through the electrode to mask or suppress the occurrence of tinnitus.

10. (canceled)

11. The method of claim 9, wherein the step of implanting a microstimulator comprises a surgical approach selected from the group consisting of through the ear canal, through a middle fossa (supra-meatal) approach, and through a mastoidectomy.

12. The method of claim 9 further comprising, before step (a) the step:
performing a test stimulation procedure comprising:
implanting a temporary electrode in the auditory system;
connecting temporarily the temporary electrode to a non-implanted stimulator; and
providing stimulation through the temporary electrode to determine the efficacy of the temporary electrode location.

13. The method of claim 12, wherein the non-implanted stimulator is worn in the inner ear.

14. The method of claim 12, wherein the non-implanted stimulator is worn behind the ear.

15. The method of claim 12, wherein the non-implanted stimulator is worn on the body.

16. The method of claim 9, wherein the step of implanting an electrode system in a pre-determined location in the auditory system is performed by implanting the electrode in the promontory of the cochlea.

17. The method of claim 9, wherein the step of implanting an electrode system in a pre-determined location in the auditory system is performed by implanting the electrode in the round window niche.
